(19) United States
(12) Patent Application Publication
(10) Pub. No.: US 2006/0083727 A1
(43) Pub. Date: Apr. 20, 2006

(54) Methods and compositions for the treatment of diseases characterized by calcification and/or plaque formation

(75) Inventors: E. Olavi Kajander, Lakeland, FL (US); K. Aho, Kuopio (FI); Neva Ciftcioglu, Houston, TX (US); H.B. Millican, Lakeland, FL (US); B. Maniscalco, Tampa, FL (US)

Correspondence Address: HOGAN & HARTSON LLP;IP GROUP, COLUMBIA SQUARE, 555 THIRTEENTH STREET, N.W., WASHINGTON, DC, 20004, US

(73) Assignee: Nanobac Pharmaceuticals, Inc.

(21) Appl. No.: 11/182,076

(22) Filed: Jul. 15, 2005

Related U.S. Application Data
(60) Provisional application No. 60/587,871, filed on Jul. 15, 2004.

(51) Int. Cl. A61K 38/54; A61K 38/43; A61K 36/87; A61K 36/734; A61K 31/525; A61K 31/65; A61K 31/714; A61K 31/198; A61K 31/455; A61K 31/4415

(52) U.S. Cl. 424/94.1; 424/94.2; 424/765; 424/766; 514/154; 514/566; 514/52; 514/251; 514/276; 514/350; 514/356; 514/565; 514/564; 424/702

(57) ABSTRACT

The invention provides methods and compositions that include a nutraceutical supplement, antibiotic, and metal chelating agent that is administered to a patient to treat or prevent pathological calcification and or plaque formation as associated with Nanobacteria Calcifying Nano-Particles and/or diseases caused there-from, The method includes the administration of a therapeutically effective nutraceutical supplement, tetracycline HCL, and ethylenediaminetetraacetic acid calcium di-sodium salt to a patient in order to prevent and treat calcific disease.

### CROSS REFERENCE TO RELATED APPLICATIONS

This application claims priority from U.S. Provisional Patent Application Ser. No. 60/587,871, filed Jul. 15, 2004, the disclosure of which is hereby incorporated by reference in its entirety.

### FIELD OF INVENTION

This invention relates, generally, to therapeutic methods and compositions for the treatment of calcification and/or plaque-based conditions and/or diseases associated with the presence of extraneous agents in human and animal blood, sera, or other fluids comprised of self-replicating calcium phosphate macromolecular complexes termed Nanobacteria or Calcifying Nano-Particles. The methods of treatment and compositions include the combination of nutritional supplements, vitamins, herbal supplements, antibiotics, and metal chelators used separately or, more preferably, in concert.

### BACKGROUND OF THE INVENTION

Biomineralization refers, generally to the formation of discrete and organized inorganic crystalline structures within macromolecular extra cellular matrices, including, for example, the formation of calcium phosphate or crystalline hydroxy apatite. Calcification is a biomineralization process in which calcium phosphate is deposited in tissue.

Examples of normal, healthy calcification include the formation of mammalian bone and dental enamel. Pathological calcification, however, has been observed to characterize a number of diseases, including but not limited to, for example, heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease. Although the cause of pathological calcification remains unknown, it has been observed that each of the foregoing conditions is often associated with the presence of a very small, mineral-associated bacteria-like self-replicating calcium-phosphate macromolecular complexes forms termed Nanobacteria (Nanobacterium sanguineum) or Calcifying Nano-Particles which are known for their ability to create calcium phosphate coated vesicles or nanoparticles that multiply in blood and in cell culture medium like living cells. Nanobacteria (“NB”) or Calcifying Nano-Particles (“CNP”) are approximately 20-200 nanometers in size and are currently the smallest known self-replicating particles or bacteria.

NB/CNP-induced calcification results from the formation of calcium-phosphate mineral deposits around each calcifying nano-particle. NB/CNPs secrete a protective calcific biofilm (i.e., a lipopolysaccharide (LPS) endotoxin biofilm) that also allows multiple NB/CNP to connect, collaborate and apparently form “colonies.” This calcific biofilm also allows the NB/CNP to expand, contract and move. The biofilm appears to be generated as part of a stress response mechanism; it is primarily observed, for example, when NB/CNP are chemically, physiologically or environmentally attacked, when they are working together and/or during NB/CNP reproduction. The biofilm that is secreted by the NB/CNP is a potent endotoxin and activates a thrombic cascade causing inflammation, swelling and the release of cytokines, interleukins, leukocytes, mast cells, collagenase, matrix metalloproteinases and other immune-response events in surrounding cells.

NB/CNP are “extremeophiles” (i.e., highly tolerant to heat, freezing, dehydration and Gamma Irradiation) and are apparently more resistant than most bacteria to destruction. Thus, NB/CNPs have been found to be residual contaminants on otherwise sterilized medical products such as tissue, blood and bovine serum. Similarly, NB/CNP cannot be killed using most antibiotics, including, for example, Penicillin, Cephalosporins, or Macrolides. It has been observed, however, that NB/CNP are sensitive to in vitro treatment with certain tetracycline's and that EDTA can assist in dissolving the protective biofilm secreted by NB/CNP.

Bench and clinical research have established that atherosclerosis is an inflammatory disease characterized by injury or infection of the vascular endothelium resulting in the formation of atheromas and pathological calcification. Inflammatory cascade responses within individual atheromas (as the immune system attempts to “wall off” or isolate an area of injury) result in the synthesis of a fibro-lipid matrix synthesis and the degradation/absorption of soft plaques. The rate of plaque synthesis-resorption is dependent upon the degree and/or stage of inflammatory activity within atheroma. Mature atheromas, for example, contain pathological calcification deposits that have been observed to increase at an annual rate of 24-82%.

Although pathological calcification deposits are a hallmark of atherosclerosis, the precise mechanism of such calcium precipitation has remained elusive. It has been widely speculated, however, that NB/CNP play a critical role in the pathological calcification processes associated with atherosclerosis. In particular, NB/CNP have been detected in atherosclerotic plaques, calcified carotid arteries, aortic aneurysms and cardiac valves. Furthermore, NB/CNP particles morphologically and functionally resemble the calcifiable vesicles, are capable of active calcium phosphate precipitation under suitable nutrient conditions and have previously been isolated from atherosclerotic aorta

Accordingly, there is a need for a specialized treatment comprising appropriate combinations of compositions of at least one of tetracycline, EDTA, and other materials for the treatment of NB/CNP-based atherosclerotic disease. A further objective of the invention is to identify a treatment protocol that is effective for treatment of atherosclerotic disease and which includes in vitro treatment with tetracycline, EDTA and other materials.

### SUMMARY OF THE INVENTION

The invention provides therapeutic methods and compositions for the treatment of calcification and/or plaque-based conditions associated with nanobacteria/calcifying nano-particle infection and in particular, atherosclerotic disease. Such therapeutic methods and compositions include administering a combination of a nutraceutical powder, tetracycline HCl and ethylenediaminetetraacetic acid disodium salt (EDTA-sequestrant).

Thus, one aspect of the invention is to provide compositions for the treatment of a disease characterized by calcification and/or plaque formation, or for the treatment of pathological calcification caused by Nanobacteria Calcifying Nano-Particles comprising at least one of a nutraceutical supplement, an antibiotic, and a metal chelator.

In accordance with the invention, the nutraceutical supplement may be comprised of a mixture of one ore more of Niacin, Vitamin B6, Folate, Vitamin C, Selenium, L-Arginine, L-Ornithine, L-Lysine, Bromelain, Trypsin, Papain, Co-Q10, Grapeseed Extract, Hawthorne Berry, Vitamin A, Vitamine E, Vitamin B1, Vitamin B2, Vitamin B12, Magnesium Citrate, Methyl Sulfonyl Methane, Curcuma Longa, Quercitin, Pycnogenol, Gugulipid.

In accordance with the invention, the antibiotic may be comprised of at least one of tetracycline, tetracycline HCL, Chlortetracycline, Democlocycline, Doxycycline, Methacycline, Oxytetracycline, Rolitetracycline, Minocycline, Sancycline, or salts thereof.

In accordance with the invention, the metal chelator may be comprised of at least one or more of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof.

In yet another aspect, the present invention relates to a method of using a composition comprising calcium chelators, bisphosphonates and/or citrate compounds which comprises administering said composition to reduce and/or prevent calcification related diseases, such as heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Keratopathy, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Pyoderma gangrenosum, Dermatomyositis, eccrine sweat duct calcification, trichoepithelioma, pilomatrixoma, necrobiosis lipoidica, Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease in an individual in need thereof.

Yet another aspect of this invention is to provide methods for administering a pharmaceutically or therapeutically effective amount of a composition of the invention to a human or mammal.

### DETAILED DESCRIPTION OF THE INVENTION

This application incorporates herein by reference in its entirety the copending and commonly assigned U.S. Non-provisional patent application Ser. No. 10/891,483 entitled “Methods and Compositions for the treatment of Diseases Characterized by Pathological Calcification” (Attorney Docket No. 19772-0004) which was filed with the United States Patent and Trademark Office on Jul. 15, 2004.

As discussed above, nanobacteria/calcifying nano-particle (“NB/CNP”) cause pathological calcification associated with a number of conditions, including atherosclerotic disease. Thus, an objective of the invention is to provide compositions useful in countering such NB/CNP-associated pathological calcification. Similarly, another objective of the invention is to provide a protocol for administering such compositions for the treatment of atherosclerotic diseases.

The invention provides therapeutic methods and compositions for the treatment of calcification and/or plaque-based conditions associated with NB/CNP infection and atherosclerotic disease. In particular, the invention includes compositions and therapeutic protocols for administering such compositions that include a nutraceutical powder, certain tetracyclines and ethylenediaminetetraacetic acid calcium disodium salt (EDTA-sequestrant). The combination of these ingredients also offers novel compositions that may be useful in the treatment of other NB/CNP related/pathological calcification conditions, including but not limited to, for example, heart or circulatory diseases such as Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

The nutraceutical powder includes Vitamin C, Vitamin B6, Niacin, Folic Acid, Selenium, EDTA, L-Arginine, L-Lysine, L-Ornithine, Bromelain, Trypsin, Niacin, CoQ10, Grapeseed Extract, Hawthorn Berry and Papain. The nutraceutical powder can also include other ingredients and materials as described below. The quantity of each component of the nutraceutical powder as well as the quantity of nutraceutical powder used in the invention may be varied for different patients and/or treatment conditions. For instance, the addition of other vitamins such as, but not limited to, Vitamin A as β Carotene, Vitamin E as d-alpha Tocopherol Succinate, Vitamin B 1 as thiamine mononitrate, Vitamin B2 as riboflavin, and Vitamin B 12 as Cyanocobalamin. Other ingredients such as Methyl Sulfonyl Methane, Magnesium Citrate, Zinc Citrate, and herbal extracts such as Mahonia aquifolium, Curcuma Longa, Quercetin, picnogenol, gugulipid, Schizandra chinensis, Licorice root, Alfalfa seed, wheatgrass, green barley grass, Chorella algae, Spirulina, Flaxseed, milk thistle, and/or Aslanguanda may be added, Other enzymes and or amino acids may also be added to the formulation such as, but not limited to, Lipase, Protease, Peptase, Serrapeptase, Cellulase, L-Glutathione.

Suitable tetracycline's include, but are not limited to, tetracycline, tetracycline HCl, chlortetracycline, demeclocycline, doxycycline, methacycline, oxytetracycline, rolitetracycline, minocycline, sancycline and pharmaceutically acceptable salts thereof. A preferred tetracycline is tetracycline HCl. The dose of these medicines may be varied for different patients and/or treatment conditions.

Suitable chelating agents include, but are not limited to one or more of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof.

One hundred patients with stable coronary artery disease (“CAD”) and positive coronary artery calcium (“CAC”) scores were initially enrolled in a four month treatment regimen that included daily administration of a three-component composition composed of the nutraceutical powder (discussed above), tetracycline HCl and ethylenediaminetetraacetic acid calcium disodium salt (EDTA-sequestrant). Exclusion criteria included: (1) known tetracycline allergy, (2) zero CAC score, (3) recent (<30 days) major adverse cardiac event, (4) women of childbearing age, (5) recent diagnosis of thyroid or parathyroid disease, (6) clinically significant renal insufficiency or liver function abnormalities and (7) recent (<30 days) acute congestive heart failure. CAC scoring was repeated at four months and serum samples were analyzed for NB/CNP antigen and baseline serology at zero, two and four months. Complete blood count, metabolic panel, liver function, C-reactive protein (hs-CRP) and lipids were analyzed at zero and four months. Other than discontinuing any herbal or vitamin preparation, patients maintained their normal medical regime during the study. Baseline History and Physical examination were performed. The same CAC scoring machine was used for each individual patient to assess initial and final CAC scores. CAC scoring radiologists were experienced in CAC scoring and were blinded to patient identity. CAC scoring was repeated after four months of treatments. Before completion of the study, one patient withdrew secondary to a presumed sensitivity to tetracycline HCL and twenty-two patients were withdrawn due to noncompliance.

As discussed in more detail below in conjunction with the accompanying Tables, 100% of the seventy-seven patients completing the study were positive for NB/CNP serology, antigen or both. Responders (n=44; 57%) had significant decreases in total CAC scores (p=0.001); the average decrease being 14%. Non-responders (n=33; 44%) had no change or had increases in CAC scores. No adverse physiologic effects were seen in the renal, hepatic, or hematopoetic systems of the treated patients. Angina was decreased or ablated in 16 of 19 patients (84%). Lipid profiles significantly improved in the non-atherogenic direction (p=0.001). Such a change in the lipid profiles is significant given that 86% of the patient group were on continuous statin medication prior to treatment.

In the accompanying Tables, data is presented as frequency and percentage distributions. Values for continuous variables are expressed as mean plus or minus a (“±”) standard deviation. Within group comparisons of initial and ending CAC scores (mean) and laboratory values were conducted using a paired t-test. Between group comparisons of continuous variables were conducted with the Student's t-test. Univariate analysis of selected discrete variables was accomplished by X², the continuity X² analysis or a two-tailed Fischer Exact test with the appropriate degrees of freedom. Statistical procedures were performed using the Number Cruncher Statistical Systems® (NCSS, Kaysville, Utah). Ap-value of less than or equal to 0.05 was designated as statistically significant in the treatment study.

Tables 1 and 2 provide statistical data and physical characteristics of participants in a study evaluating the clinical effects of the invention. In Table 1, the initial physical and clinical characteristics of the final study participants (n=77) are described. In Table 2, the seventy-seven participants are subdivided into “responder” and “nonresponder” groups (as defined below) based on their response to treatment with the invention. Table 2 further illustrates the pretreatment physical characteristics for both the “responder” and “nonresponder” groups. The data in Table 2 indicates that both groups had comparable pre-treatment clinical variables and risk factors.

Table 3 demonstrates that 44 (57%) of the seventy-seven patients responded to treatment with the invention as evidenced by a decrease in total CAC score. The remaining 33 patients (43%) were considered “nonresponders” based solely on their CAC score. As can be seen in Table 3, total CAC scores decreased significantly (p=0.001) from the beginning to the end of the study for the responder group. Significant reduction in both the left anterior descending coronary artery and the right coronary artery CAC scores were also documented p=0.002). There was no significant difference found in the left main coronary artery (p=0.972) or circumflex coronary artery CAC scores (p=0.106).

Table 4 illustrates that all responder group patients tested positive for the presence of anti-NB/CNP IgG antibodies prior to the commencement of therapy. During treatment with the invention, NB/CNP antigen and serology titers tended to fluctuate (although the fluctuations were not statistically significant) in all patients independent of changes in CAC scores or stage of therapy.

Table 5 demonstrates the beneficial changes in the lipid profiles for responder group patients following treatment with the invention. Notably, it was observed that responder group patients experienced reduced total cholesterol levels (p=0.001), reduced triglycerides p=0.006), decreased LDL (p=0.001) and increased HDL (p=0.001) following treatment with the invention.

In addition to the favorable results illustrated in Tables 1-5, other data supports the efficacy of the invention. For example, prior to treatment, 19 patients (25%) had stable angina pectoris. Following four months of treatment, the angina symptoms had been either eliminated or substantially ameliorated in 16 of the 19 (84%) patients (p=0.013). Similarly, two patients (3%) with severe claudication and faint pedal pulses reported a diminution of claudication symptoms and the return of their peripheral pulses to normal values following treatment.

The foregoing data demonstrates that administration of a combination of a nutraceutical powder, certain antibiotics and EDTA for sustained periods is effective for treating CAD patients. Specifically, every second CAD patient treated as described herein demonstrated an objective improvement in their cardiac vasculature performance and had appreciably decreased CAC scores (avg. ˜14% decrease). These results are particularly encouraging considering that CAC scores are known to increase by more than 20% annually. These results highlight the significance of the invention given that there have been no previous reports showing a significant decrease in CAC scores pursuant to any known means of intervention.

Furthermore, based on the foregoing data (for both the responder and nonresponder patient groups), it is possible to infer that other variables, including, for example, treatment time, plaque density/volume, tissue penetration and blood supply may be critical factors that influence overall outcomes related to treatment efficacy. Based on these findings, it appears that CAC scores would continue to decrease in conjunction over longer periods of therapy (i.e., plaque regression over time). Additionally, the treatment regimen did not produce any apparent or significant adverse physiological effects on the study participants.

### EXAMPLES

The invention is further illustrated by the following examples. All scientific and technical terms have the meanings as understood by one with ordinary skill in the art. The specific examples that follow illustrate the methods in which the compositions of the present invention may be prepared and/or protocols for the administration of such compositions to a patient in need thereof. Such examples, however, are merely illustrative and are not intended nor should be construed as limiting the invention in sphere or scope. The methods may be adapted and/or varied in order to produce compositions embraced by this invention but not specifically disclosed. Further, variations of the methods to produce the same compositions in somewhat different fashion will be evident to one skilled in the art.

1. Formulations

In one embodiment of the invention, a composition for treatment of atherosclerotic diseases associated with NB/CNP infection that includes at least three components is disclosed. These components include a quantity of a nutraceutical powder (that includes Vitamin C, Vitamin B6, Niacin, Folic Acid, Selenium, EDTA, L-Arginine, L-Lysine, L-Ornithine, Bromelain, Trypsin, Niacin, CoQ10, Grapeseed Extract, Hawthorn Berry and Papain), a quantity of a tetracycline compound and a quantity of ethylenediaminetetraacetic acid disodium salt or calcium di-sodium salt (EDTA-sequestrant).

In another embodiement, the nutraceutical powder may also include other Vitamins such as, but not limited to, Vitamin A, Vitamin E, Vitamin B1, B2, and B12. Materials such as methyl sulfonyl methane (MSM), Citrates such as Magnesium Citrate or Zinc Citrate and herbal extracts such as Mahonia aquifolium, Curcuma longa (turmeric), Lipase, Protease, Peptase, Serrapeptase, Cellulase, L-Glutathione, Schizandra Chinensis, Licorice Root, Quercetin, Alfalfa Seed, Wheatgrass, Green Barley Grass, Chlorella Algae, Spirulina, Flaxseed, Milk Thistle, picnogenol, Gugulipid, Aslaguanda may also be added to the formulae as predicated by specific patient requirements.

In another embodiment, the quantity of the nutraceutical powder component is mixed with water, juice (e.g., apple or orange juice) or other suitable liquid prior to being administered.

In another embodiment, the quantity of the nutraceutical powder component is 5 cm³ and is mixed with water, juice (e.g., apple or orange juice) or other suitable liquid prior to being administered.

In other embodiments, the quantity of nutraceutical powder is formulated as either a pill or capsule.

In another embodiment the tetracycline compound is tetracycline HCl.

In another embodiment, 500 mg of the tetracycline HCl component is formulated as a capsule before being administered.

In another embodiment, 500 mg of the tetracycline HCl component is formulated as a pill before being administered.

In another embodiment, 1500 mg of the ethylenediaminetetraacetic acid calcium disodium salt (EDTA-sequestrant) component is formulated as a suppository before being administered.

As will be appreciated by those knowledgeable in the art, the therapeutic components of the invention may be individually or collectively formulated in different manners, quantities and/or combinations and may otherwise be used in combination with other treatments. Furthermore, the therapeutic composition of the present invention may be packaged in any convenient, appropriate packaging.

In addition to the specific component formulations recited in the above examples, each component of the invention may be in various other forms suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions) for administration by inhalation (for example as a finely divided powder or a liquid aerosol), for administration by insufflation (for example as a finely divided powder) or for parenteral administration (for example as a sterile aqueous or oily solution for intravenous, subcutaneous, or intramuscular dosing), or as a suppository for rectal dosing.

Suitable pharmaceutically-acceptable excipients for a tablet formulation include, for example, inert diluents such as lactose, sodium carbonate, granulating and disintegrating agents such as corn starch or alginic acid; binding agents such as starch; lubricating agents such as stearate, stearic acid, fumed silica or talc; preservative agents such as ethyl or propyl p-hydroxybenzoate, and anti-oxidants, such as ascorbic acid or tocopherol acetate. Tablet formulations may be uncoated or coated either to modify their disintegration and the subsequent absorption of the active ingredient within the gastrointestinal tract, or to improve their stability and/or appearance, in either case, using conventional coating agents or fillers such as hydroxy propyl methyl cellulose (Methocel) or other cellulose and procedures well known in the art

Compositions for oral use of one or more of the components may be in the form of hard gelatin capsules in which the active ingredient is mixed with an inert solid diluent, for example, calcium carbonate, calcium sulfate dihydrate, calcium phosphate or kaolin, or as soft gelatin capsules in which the active ingredient is mixed with water or an oil such as peanut oil, liquid paraffin, or olive oil.

Aqueous suspensions of one or more of the components generally contain the active ingredient in finely powdered form together with one or more suspending agents, such as sodium carboxymethylcellulose, methylcellulose, hydroxyethyl starch, starch acetate, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid or tocopherol acetate), coloring agents, flavoring agents, and/or sweetening agents (such as sucrose, stevia, sucralose, xylitol, saccharine or aspartame).

Oily suspensions of one or more of the components may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid or tocopherol acetate.

Dispersible powders and granules suitable for preparation of an aqueous suspension of one or more of the components by the addition of water (or other suitable liquid such as juice) generally contain the recited ingredient(s) together with a dispersing or wetting agent, suspending agent and one or more preservatives. Suitable dispersing or wetting agents and suspending agents are exemplified by those already mentioned above. Additional excipients such as sweetening, flavoring and coloring agents may also be present.

One or more of the components of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs of one or more of the components may be formulated with sweetening agents such as glycerol, propylene glycol, sorbitol, aspartame or sucrose, and may also contain a demulcent, preservative, flavoring and/or coloring agent.

One or more of the components may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations of one or more of the components may be prepared by mixing the active ingredient with a suitable non-irritating excipient which is solid at ordinary temperatures but liquid at the rectal temperature and will therefore melt in the rectum to release the drug. Suitable excipients include, for example, cocoa butter, polyethylene glycols and stearates.

Compositions of one or more of the components for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions of one or more of the components for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

The amount of one or more of the ingredients comprising each component of the invention can be altered or combined with one or more excipients to produce a single dosage form and each such combination may vary depending upon the host treated and the particular route of administration. For example, a formulation intended for oral administration to humans may contain a component compounded with an appropriate and convenient amount of excipients that may vary from about 0.1 to about 99% by weight of the total composition. Such dosages may be obtained by mixing each component of the invention with different excipients (as recited above) such as agglutinants, disintegrators, lubricants, sliders or fillers. Other excipients include lactose, corn starch, saccharose, stearate, microcrystalline cellulose, sodium croscarmellose gelatin, cellulose acetophtalate, titanium dioxide, fumed and precipitated silicates, special talc for tablets and polyethylene glycol.

2. Treatment Protocols

The invention further contemplates a protocol for administering the three-components of the invention for treatment of atherosclerotic diseases associated with NB/CNP infection. According to this aspect of the invention, there is provided a protocol for the separate and sequential administration of the nutraccutical powder (discussed above), tetracycline HCl and ethylenediaminetetraacetic acid disodium salt (EDTA-sequestrant) in sufficient quantity to an individual in need thereof. The protocol of the present invention can be administered to a patient by any available and effective delivery system including, but not limited to, oral, parenteral, transdermal, intranasal, sublingual, transmucosal, intra-arterial, or intradermal modes of administration in dosage unit formulations containing conventional nontoxic pharmaceutically acceptable carriers, adjuvants, and vehicles as desired, such as a depot or a controlled release formulation.

In one embodiment of the treatment protocol, a patient is instructed, prior to going to bed, to mix approximately 5 cm³ of the nutraceutical powder in water, juice (e.g., apple or orange juice) or other suitable liquid prior to being administered. Thereafter, the patient is instructed to orally consume the nutraceutical powder solution. In this embodiment, the patient is also instructed to orally consume approximately 500 mg of tetracycline HCl that had been formulated as a capsule before administration. Next, the patient is instructed to rectally insert approximately 1500 mg of ethylenediaminetetraacetic acid disodium salt (EDTA-sequestrant) that had been formulated as a suppository before administration. Once the three components of the composition were administered, the patient was instructed to lie down flat and fall asleep.

Variations in the above treatment protocol can readily be made. In other embodiments, for example, the order in which the components are administered can be altered. Similarly, in differing embodiments, different quantities of each component may be employed and/or the components may individually or collectively formulated in different manners as warranted by prevailing conditions or patient needs.

TABLE 1
Study Variables

| Variables | | Number | Percentage |
|---|---|---|---|
| Total Study | | 77 | 100 |
| Number | | | |
| Gender | Male | 62 | 80.5 |
| | Female | 15 | 19.5 |
| Age Groups | Under 50 | 4 | 5.2 |
| (years) | 50-59 | 25 | 32.5 |
| | 60-69 | 25 | 32.5 |
| | 70-79 | 20 | 26 |
| | 80 and Over | 3 | 3.9 |
| | Mean | 63.2 ± 8.9 | |
| | Range | 42-81 | |
| Coronary Risk | Hypertension | 52 | 67.5 |
| Factors | Hyperlipidemia | 68 | 88.3 |
| | Diabetes Mellitus | 20 | 26.0 |
| | Peripheral Vascular Disease | 10 | 13.0 |
| | History of Congestive Heart | 5 | 6.5 |
| | Renal Insufficiency | 2 | 2.6 |
| | (Creatine > 2.0 mg/dL) | | |
| | Previous Myocardial Infarction | 14 | 18.1 |
| | Stable Angina | 19 | 24.7 |
| Previous | Prior Coronary Artery Bypass | 39 | 50.7 |
| Cardiovascular | Grafting | | |
| | Prior Percutaneous Coronary | 17 | 22.1 |
| | Intervention | | |
| Current | Statins | 66 | 85.7 |
| Medications | Nitrates | 23 | 29.9 |
| | Anticagulants | 3 | 3.9 |
| | Beta Blockers | 42 | 54.6 |
| | ACE Inhibitors | 28 | 36.4 |
| | Diuretics | 20 | 26.0 |
| | Antiplatelets | 55 | 71.4 |
| | Calcium Blockers | 12 | 15.6 |
| | ARB | 18 | 23.4 |

TABLE 2
Comparison of Pretreatment Clinical Variables and
Risk Factors of Study Population by Patient Group

| | | Responders | | NonResponders | | |
|---|---|---|---|---|---|---|
| Variables | | Number/(Percentage) | | Number/(Percentage) | | p Value |
| Total Study | | 44 | (100) | 33 | (100) | |
| Number | | | | | | |
| Gender | Male | 37 | (84.1) | 25 | (75.8) | 0.361 |
| | Female | 7 | (15.9) | 8 | (24.2) | |
| Age/Groups | Mean | 64.9 ± 8.9 | | 61.0 ± 8.4 | | 0.058 |
| | Range | 48-81 | | 42-80 | | |
| | Under 50 | 2 | (4.5) | 2 | (6.1) | |
| | 50-59 | 12 | (27.3) | 13 | (39.4) | |
| | 60-69 | 14 | (31.8) | 11 | (33.3) | |
| | 70-79 | 14 | (31.8) | 6 | (18.2) | |
| | 80 and Over | 2 | (4.5) | 1 | (3.0) | |
| Coronary Risk | Hypertension | 31 | (70.5) | 21 | (63.6) | 0.527 |
| Factors | Hyperlipidemia | 41 | (93.2) | 27 | (81.8) | 0.160 |
| | Diabetes Mellitus | 11 | (25.0) | 9 | (27.3) | 0.822 |
| | Peripheral Vascular | 4 | (9.1) | 6 | (18.2) | 0.311 |
| | Disease | | | | | |
| | History of Congestive | 3 | (6.8) | 2 | (6.1) | 0.999 |
| | Heart Failure | | | | | |
| | Renal Insufficiency | 1 | (2.3) | 1 | (3.0) | 0.999 |
| | (Creatine > 2.0 | | | | | |
| | Previous Myocardial | 10 | (22.8) | 4 | (12.1) | 0.370 |
| | Stable Angina | 16 | (36.4) | 3 | (9.1) | 0.013 |
| Previous | Prior Coronary Artery | 24 | (54.5) | 15 | (45.5) | 0.430 |
| Cardiovascular | Bypass Grafting | | | | | |
| Intervention | Prior Percutaneous | 10 | (22.7) | 7 | (9.1) | 0.999 |
| | Coronary Intervention | | | | | |
| Current | Statins | 39 | (88.6) | 27 | (81.8) | 0.515 |
| Medications | Nitrates | 16 | (36.4) | 7 | (21.2) | 0.151 |
| | Anticagulants | 2 | (4.5) | 1 | (3.0) | 0.999 |
| | Beta Blockers | 27 | (61.4) | 15 | (45.5) | 0.165 |
| | ACE Inhibitors | 15 | (34.1) | 13 | (39.4) | 0.632 |
| | Diuretics | 12 | (27.3) | 8 | (24.2) | 0.764 |
| | Antiplatelets | 32 | (72.7) | 23 | (69.7) | 0.771 |
| | Calcium Blockers | 5 | (11.4) | 7 | (21.2) | 0.238 |
| | ARB | 8 | (18.2) | 10 | (30.3) | 0.214 |

TABLE 3
Comparison of Initial and Ending
CAC Scan Scores for Responders

| Variables | Initial Score | Ending Score | p Value |
|---|---|---|---|
| Total Responder Number | 44 (100) | 44 (100) | |
| Total Score | 2033.0 | 1753.8 | 0.001 |
| Left Coronary Artery | 89.5 | 89.8 | 0.972 |
| Left Anterior | 927.7 | 788.4 | 0.002 |
| Descending Coronary | | | |
| Artery | | | |
| Circumflex Coronary | 244.2 | 211.1 | 0.106 |
| Artery | | | |
| Right Coronary Artery | 771.0 | 664.5 | 0.002 |

TABLE 4
Comparison of Responder NB/CNP Antibody Levels (Units) and Antigen Levels (Units)

| | | Beginning | Two-Month | | |
|---|---|---|---|---|---|
| | | Value | Value | EndingValue | |
| Variables | | (Mean ± S.D.) | (Mean ± S.D.) | (Mean ± S.D.) | p Value |
| NB/CNP | | 0.864 ± 0.35 | 0.954 ± 0.485 | 0.981 ± 0.50 | |
| Antibody | | | | | |
| | Beginning Value v. | | | | 0.269 |
| | Two Month Value | | | | |
| | Beginning Value v. | | | | 0.300 |
| | Ending Value | | | | |
| | Two-Month Value v. | | | | 0.799 |
| | Ending Value | | | | |
| NB/CNP | | 2.045 ± 4.64 | 2.067 ± 4.30 | 4.012 ± 8.39 | |
| Antigen | | | | | |
| | Beginning Value v. | | | | 0.982 |
| | Two Month Value | | | | |
| | Beginning Value v. | | | | 0.206 |
| | Ending Value | | | | |
| | Two-Month Value v. | | | | 0.219 |
| | EndingValue | | | | |

TABLE 5
Comparison of Responders Beginning
and Ending Cholesterol Levels

| | Beginning Value | EndingValue | |
|---|---|---|---|
| | (Mean ± S.D.) | (Mean ± S.D.) | |
| Lipid Panel | (mmol/L) | (mmol/L) | p Value |
| Total Cholesterol | 4.9 ± 1.2 | 4.2 ± 0.88 | 0.001 |
| Triglycerides | 2.5 ± 3.2 | 1.9 ± 2.3 | 0.006 |
| HDL Cholesterol | 1.2 ± 0.319 | 1.4 ± 0.34 | 0.001 |
| LDL Cholesterol | 2.6 ± 0.9 | 2.1 ± 0.8 | 0.001 |
| | Beginning Value | EndingValue | |
| | (Mean ± S.D.) | (Mean ± S.D.) | |
| Lipid Panel | (mg/dL) | (mg/dL) | p Value |
| Total Cholesterol | 188.6 ± 47.4 | 164.5 ± 33.8 | 0.001 |
| Triglycerides | 232.0 ± 302.5 | 179.4 ± 221.1 | 0.006 |
| HDL Cholesterol | 47.4 ± 12.1 | 52.25 ± 13.0 | 0.001 |
| LDL Cholesterol | 101.8 ± 35.6 | 81.3 ± 29.5 | 0.001 |

1. A composition for the treatment of a disease characterized by calcification and or plaque formation comprising at least one of;
a. a nutraceutical supplement,
b. antibiotic, and
c. a metal chelator.

2. A composition for the treatment of pathological calcification caused by Nanobacteria Calcifying Nano-Particles comprising therapeutic agents in effective amounts comprising at least one of;
a. a nutraceutical supplement
b. an antibiotic, and a
c. a metal chelator.

3. The composition of claim 2, wherein said composition is administered to a patient daily for a period of greater than 3 months for the treatment of at least one of coronary artery disease, atherosclerosis or arteriosclerosis.

4. The composition of claim 2, wherein said diseases include: Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

5. The composition of claim 2, wherein said nutraceutical supplement is comprised of a mixture of at least one of the following in doses effective for the treatement of disease characterized by calcification and or plaque formation: Niacin, Vitamin B6, Folate, Vitamin C, Selenium, L-Arginine, L-Ornithine, L-Lysine, Bromelain, Trypsin, Papain, Co-Q10, Grapeseed Extract, Hawthorne Berry, Vitamin A, Vitamine E, Vitamin B1, Vitamin B2, Vitamin B12, Magnesium Citrate, Methyl Sulfonyl Methane, Curcuma Longa, Quercitin, Pycnogenol, Gugulipid.

6. A composition for the treatment of pathological calcification caused by Nanobacteria Calcifying Nano-Particles comprising therapeutic agents in effective amounts comprising at least one of:
a. a nutraceutical supplement comprising at least one of Vitamin C, Vitamin B6, Niacin, Folic Acid, Selenium, EDTA, L-Arginine, L-Lysine, L-Ornithine, Bromelain, Trypsin, CoQ10, Grapeseed Extract, Hawthorn Berry and Papain;
b. an antibiotic, and
c. a metal chelator.

7. A nutraceutical composition comprising at least one of Vitamin C, Vitamin B6, Niacin, Folic Acid, Selenium, EDTA, L-Arginine, L-Lysine, L-Ornithine, Bromelain, Trypsin, CoQ10, Grapeseed Extract, Hawthorn Berry and Papain.

8. The composition of claim 2, wherein said nutraceutical is in the form of an oral dispersible powder or granule, compressed pill or tablet, hard or soft capsule, suspension, lozenges, aqueous or oily suspensions, emulsions, syrup or elixir, or sublingual solution.

9. The composition of claim 2, wherein said nutraceutical is in the form of a topical cream, ointment, gel, aqueous solution, aqueous suspension, oil based solution or oil based suspension.

10. The composition of claim 2, wherein said nutraceutical is in the form of a finely divided powder or liquid aerosol to be inhaled or insufflated.

11. The composition of claim 2, wherein said nutraceutical is in the form of a sterile aqueous or oil based solution for parenteral administration such as intravenous, subcutaneous, or intramuscular dosing.

12. The composition of claim 6, wherein said nutraceutical supplement is in the form of a powder and is mixed with juice or water and taken once daily by a patient.

13. A composition of nutraceutical powder comprising at least one of Vitamin C, Vitamin B6, Niacin, Folic Acid, Selenium, EDTA, L-Arginine, L-Lysine, L-Ornithine, Bromelain, Trypsin, Niacin, CoQ10, Grapeseed Extract, Hawthorn Berry and Papain in a powder form that is dispersed with water or juice and consumed by a patient in order to prevent calcification and the formation of plaque in the body as associated with nanobacteria calcifying nano-particles.

14. The composition of claim 13, wherein said nutraceutical is taken in combination with an antibiotic and metal chelating agent.

15. A composition for the treatment of pathological calcification caused by Nanobacteria Calcifying Nano-Particles comprising therapeutic agents in effective amounts comprising at least one of:
a. a nutraceutical supplement
b. an antibiotic comprised of at least one of tetracycline, tetracycline HCL, Chlortetracycline, Democlocycline, Doxycycline, Methacycline, Oxytetracycline, Rolitetracycline, Minocycline, Sancycline, or salts thereof, and
c. a metal chelator.

16. A composition for the treatment of pathological calcification caused by Nanobacteria Calcifying Nano-Particles comprising therapeutic agents in effective amounts comprising at least one of:
a. a nutraceutical supplement
b. tetracycline HCl, and
c. a metal chelator.

17. The composition of claim 15, wherein the dosage of said antibiotic is between 250-2000 mg per day per patient.

18. The composition of claim 15, wherein the dosage of said antibiotic is between 300-1000 mg per day per patient.

19. The composition of claim 15, wherein the dosage of said antibiotic is 500 mg per day per patient.

20. The composition of claim 15, wherein said antibiotic is the form of an oral dispersible powder or granule, compressed pill or tablet, hard or soft capsule, suspension, lozenges, aqueous or oily suspensions, emulsions, syrup, elixir or sublingual solution.

21. The composition of claim 15, wherein said antibiotic is in the form of a topical cream, ointment, gel, aqueous solution, aqueous suspension, oil based solution or oil based suspension.

22. The composition of claim 15, wherein said antibiotic is in the form of a finely divided powder or liquid aerosol to be inhaled or insufflated.

23. The composition of claim 15, wherein said antibiotic is in the form of a sterile aqueous or oil based solution for parenteral administration such as intravenous, subcutaneous, or intramuscular dosing.

24. An oral dose of Tetracycline HCL in a dosage of 500 mg per day as taken by a patient in a therapeutically effective amount to stop replication of nanobacteria calcifying nano-particles in patients suffering from calcific disease or plaque.

25. A composition for the treatment of pathological calcification caused by Nanobacteria Calcifying Nano-Particles comprising therapeutic agents in effective amounts comprising at least one of:
a. a nutraceutical supplement;
b. an antibiotic; and
c. a metal chelator, comprising of at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof including ethylenediaminetetraacetic acid di-sodium salt or calcium di-sodium salt heavy metal sequesterant.

26. The composition of claim 25, wherein said metal chelator is in the form of a topical cream, ointment, gel, aqueous solution, aqueous suspension, oil based solution or oil based suspension.

27. The composition of claim 25, wherein said metal chelator is in the form of an oral dispersible powder or granule, compressed pill or tablet, hard or soft capsule, suspension, lozenges, aqueous or oily suspensions, emulsions, syrup, elixir or sublingual solution.

28. The composition of claim 25, wherein said metal chelator is in the form of a controlled, prolonged, extended, or sustained release composition.

29. The composition of claim 25, wherein said metal chelator is in the form of an enteric coated tablet, pill, or hard/soft capsule.

30. The composition of claim 25, wherein said metal chelator is in the form of a finely divided powder or liquid aerosol to be inhaled or insufflated.

31. The composition of claim 25, wherein said metal chelator is in the form of a sterile aqueous or oil based solution for parenteral administration such as intravenous, subcutaneous, or intramuscular dosing.

32. The composition of claim 25, wherein said metal chelator is in the form of a suppository.

33. The composition of claim 25, wherein the daily dosage is 100-2000 mg per day per patient.

34. The composition of claim 25, wherein the daily dosage is 1500 mg per day per patient.

35. Ethylenediaminetetraacetic acid calcium di-sodium salt in a daily dosage is 100-2000 mg per day per patient delivered to a patient in the form of a suppository wherein said patient applies said suppository and is instructed to lay flat and go to sleep.

36. The composition of claim 35, wherein said suppository is used in concert with a nutraceutical supplement and antibiotic to treat disease as caused by calcification and or plaque formation.

37. A composition for the treatment of diseases characterized by pathologic calcification comprising a mixture of an antibiotic and chelating agent.

38. The composition of claim 37, wherein said mixture is in the form of an oral dispersible powder or granule, compressed pill or tablet, hard or soft capsule, suspension, lozenges, aqueous or oily suspensions, emulsions, syrup, elixir or sublingual solution.

39. The composition of claim 37, wherein said metal chelator is in the form of an orally administered controlled, prolonged, extended, or sustained release composition.

40. The composition of claim 37, wherein said metal chelator is in the form of an orally administered enteric coated tablet, pill, or hard/soft capsule.

41. The composition of claim 37, wherein said mixture is in the form of a finely divided powder or liquid aerosol to be inhaled or insufflated.

42. The composition of claim 37, wherein said mixture is in the form of a sterile aqueous or oil based solution for parenteral administration such as intravenous, subcutaneous, or intramuscular dosing.

43. The composition of claim 37, wherein said mixture is in the form of a suppository.

44. The composition of claim 37, wherein said antibiotic is selected from at least one of: tetracycline, testracycline HCL, Chlortetracycline, Democlocycline, Doxycycline, Methacycline, Oxytetracycline, Rolitetracycline, Minocycline, Sancycline, or salts thereof and said chelating agent is selected from at least one of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof including ethylenediaminetetraacetic acid di-sodium salt or calcium di-sodium salt heavy metal sequesterant.

45. The composition of claim 44, wherein said EDTA salt is in a dose of between 100 to 2000 mg per day and said Tetracycline HCl is in a dose of between 250-3000 mg per day.

46. The composition of claim 44, wherein said EDTA is in a dose of 1500 mg per day and said Tetracycline is in a dose of 500 mg per day.

47. An oral dispersible powder or granule, compressed pill or tablet, hard or soft capsule, suspension, lozenges, aqueous or oily suspensions, emulsions, syrup, elixir or sublingual solution comprising EDTA and Tetracycline for the treatment of Coronary Artery Disease as caused by pathologic calcification.

48. A method for the treatment of diseases characterized by pathologic calcification comprising administering a therapeutically effective amount of a composition comprising at least one of:
a. a nutraceutical supplement comprising at least one of Vitamin C, Vitamin B6, Niacin, Folic Acid, Selenium, EDTA, L-Arginine, L-Lysine, L-Ornithine, Bromelain, Trypsin, Niacin, CoQ10, Grapeseed Extract, Hawthorn Berry and Papain;
b. Tetracycline HCl; and
c. Ethylenediaminetetraacetic acid calcium di-sodium salt.

49. The method of claim 48, wherein said nutraceutical is in the form of a powder mixed with water or suitable liquid and is administered at 5 cc per day.

50. The method of claim 48, wherein said Tetracycline HCl is in the form of a capsule and a dosage of 500 mg per day.

51. The composition of claim 48, wherein said ethylenediaminetetraacetic acid calcium di-sodium salt is in the form of a suppository and administered at 1500 mg per day.

52. A method for treating coronary artery disease caused by calcification and or plaque formation comprising administering a pharmaceutically effective amount of a composition comprising at least one of:
a. a nutraceutical supplement comprising at least one of Vitamin C, Vitamin B6, Niacin, Folic Acid, Selenium, EDTA, L-Arginine, L-Lysine, L-Ornithine, Bromelain, Trypsin, Niacin, CoQ10, Grapeseed Extract, Hawthorn Berry and Papain;
b. Tetracycline HCL; and
c. Ethylenediaminetetraacetic acid calcium d-sodium salt.

53. The method of claim 52, wherein the compositions are administered for the treatment of at least one of the following diseases:
Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

54. The method of claim 52, wherein said nutraceutical supplement is taken in powder form as mixed with water at a dose of 5 cubic centrimeters, said Tetracycline is taken in capsule form at a dose of 500 mg, and said ethylenediamineacetic acid calcium di-sodium salt is taken in rectal suppository form at a dose of 1500 mg in order to treat diseases caused by calcification and or plaque formation.

55. A method for treating or preventing the growth of nanobacteria calcifying nanoparticles in vivo, comprising administering a therapeutically effective amount of a composition comprising calcium chelators, antibiotics, and nutraceutical supplements.

56. The method of claim 55, wherein said calcium chelator is ethylenediaminetetraacetic acid calcium disodium salt at a dosage of between 100-2000 mg and administered in the form of a suppository daily for at least 3 months.

57. The method of claim 55, wherein said antibiotic is tetracycline HCL at a dosage of 250-750 mg and administered in the form of a tablet daily for at least 3 months.

58. The method of claim 55, wherein said nutraceutical supplement is at a dosage of 5 cubic centimeters per day and administered in the form of a powder.

59. The method of treating a disease characterized by calcification and or plaque formation comprising administering to a patient a combination comprised of at least one of:
a. a nutraceutical supplement;
b. antibiotic; and
c. a metal chelator.

60. A method of treating pathological calcification caused by Nanobacteria Calcifying Nano-Particles comprising administering to a patient therapeutic agents in effective amounts comprising at least one of:
a. a nutraceutical supplement;
b. an antibiotic; and
c. a metal chelator.

61. The method of claim 60, wherein said composition is administered to a patient daily for a period of greater than 3 months for the treatment of at least one of coronary artery disease, atherosclerosis or arteriosclerosis.

62. The method of claim 60, wherein said diseases include: Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

63. A method of treating pathological calcification caused by Nanobacteria Calcifying Nano-Particles, comprising administering to a patient therapeutic agents in effective amounts comprising at least one of:
a. a nutraceutical supplement;
b. an antibiotic, comprised of at least one of tetracycline, testracycline HCL, Chlortetracycline, Democlocycline, Doxycycline, Methacycline, Oxytetracycline, Rolitetracycline, Minocycline, Sancycline, or salts thereof; and
c. a metal chelator.

64. The method of claim 63, wherein said antibiotic is tetracycline HCl.

65. The method of claim 64, wherein the dosage of said antibiotic is between 250-2000 mg per day per patient.

66. The method of claim 64, wherein the dosage of said antibiotic is between 300-1000 mg per day per patient.

67. The method of claim 64, wherein the dosage of said antibiotic is 500 mg per day per patient.

68. The method of claim 63, wherein said antibiotic is the form of an oral dispersible powder or granule, compressed pill or tablet, hard or soft capsule, suspension, lozenges, aqueous or oily suspensions, emulsions, syrup, elixir or sublingual solution.

69. The method of claim 63, wherein said antibiotic is in the form of a topical cream, ointment, gel, aqueous solution, aqueous suspension, oil based solution or oil based suspension.

70. A method for the treatment of diseases characterized by pathologic calcification comprising administering a therapeutically effective amount of a composition comprising at least one of:
a. a nutraceutical supplement comprising at least one of Vitamin C, Vitamin B6, Niacin, Folic Acid, Selenium, EDTA, L-Arginine, L-Lysine, L-Ornithine, Bromelain, Trypsin, Niacin, CoQ10, Grapeseed Extract, Hawthorn Berry and Papain;
b. Tetracycline HCl; and
c. Ethylenediaminetetraacetic acid calcium di-sodium salt.

71. The method of claim 70, wherein said nutraceutical is in the form of a powder mixed with water or suitable liquid and is administered at 5 cc per day.

72. The method of claim 70, wherein said Tetracycline HCl is in the form of a capsule and a dosage of 500 mg per day.

73. The method and composition of claim 70, wherein said ethylenediaminetetraacetic acid calcium di-sodium salt is in the form of a suppository and administered at 1500 mg per day.

74. A method for treating coronary artery disease caused by calcification and or plaque formation comprising administering to a patient a therapeutically effective amount of at least one of:
a. A nutraceutical supplement comprising at least one of Vitamin C, Vitamin B6, Niacin, Folic Acid, Selenium, EDTA, L-Arginine, L-Lysine, L-Ornithine, Bromelain, Trypsin, Niacin, CoQ10, Grapeseed Extract, Hawthorn Berry and Papain;
b. Tetracycline HCL; and
c. Ethylenediaminetetraacetic acid calcium d-sodium salt.

75. The method of claim 74, wherein the compositions are administered for the treatment of at least one of the following diseases;
Arteriosclerosis, Atherosclerosis, Coronary Heart Disease, Chronic Heart Failure, Valve Calcifications, Arterial Aneurysms, Calcific Aortic Stenosis, Transient Cerebral Ischemia, Stroke, Peripheral Vascular Disease, Monckeberg's Disease, Vascular Thrombosis; Dental Diseases such as Dental Plaque, Gum Disease (dental pulp stones), calcification of the dentinal papilla, and Salivary Gland Stones; Chronic Infection Syndromes such as Chronic Fatigue Syndrome; Kidney and Bladder Stones, Gall Stones, Pancreas and Bowel Diseases such as Pancreatic Duct Stones, Crohn's Disease, Colitis Ulcerosa; Blood disorders; Adrenal Calcification; Liver Diseases such as Liver Cirrhosis and Liver Cysts; Testicular Microliths, Chronic Calculous Prostatitis, Prostate Calcification, Calcification in Hemodialysis Patients, Malacoplakia; Autoimmune Diseases such as Lupus Erythematosous, Schleroderma, Dermatomyositis, Cutaneous polyarteritis, Panniculitis (Septal and Lobular), Antiphospholipid Syndrome, Arteritis Nodosa, Thrombocytopenia, Hemolytic Anemia, Myelitis, Livedo Reticularis, Chorea, Migraine, Junvenile Dermatomyositis, Graves Disease, Chronic Thyroiditis, Hypothyreoidism, Type 1 Diabetes Mellitis, Addison's Disease, and Hypopituitarism; Placental and Fetal Disorders, Polycystic Kidney Disease, Glomerulopathies; Eye Diseases such as Corneal Calcifications, Cataracts, Macular Degeneration and Retinal Vasculature-derived Processes and other Retinal Degenerations; Retinal Nerve Degeneration, Retinitis, and Iritis; Ear Diseases such as Otosclerosis, Degeneration of Otoliths and Symptoms from the Vestibular Organ and Inner Ear (Vertigo and Tinnitus); Thyroglossal cysts, Thyroid Cysts, Ovarian Cysts; Cancer such as Meningiomas, Breast Cancer, Prostate Cancer, Thyroid Cancer, Serous Ovarian Adenocarcinoma; Skin diseases such as Calcinosis Cutis, Skin Stones, Calciphylaxis, Psoriasis, Eczema, Lichen Ruber Planus or Lichen Simple Cysts; Choroid Plexus Calcification, Neuronal Calcification, Calcification of the Falx Cerebri, Calcification of the Intervertebral Cartilage or Disc, Intercranial or Cerebral Calcification, Rheumatoid Arthritis, Calcific Tenditis, Oseoarthritis, Fibromyalgia, Bone Spurs, Diffuse Interstitial Skeletal Hyperostosis, Intracranial Calcifications such as Degenerative Disease Processes and Dementia; Erythrocyte-Related Diseases involving Anemia, Intraerythrocytic Nanobacterial Infection and Splenci Calcifications; Chronic Obstructive Pulmonary Disease, Broncholiths, Bronchial Stones, Neuropathy, Calcifications and Encrustations of Implants, Mixed Calcified Biofilms, and Myelodegenerative Disorders such as Multiple Sclerosis, Lou Gehrig's, and Alzheimer's Disease.

76. The method of claim 74, wherein said nutraceutical supplement is taken in powder form as mixed with water at a dose of 5 cubic centrimeters, said Tetracycline is taken in capsule form at a dose of 500 mg, and said ethylenediamineacetic acid calcium di-sodium salt is taken in rectal suppository form at a dose of 1500 mg in order to treat diseases caused by calcification and or plaque formation.

77. A composition comprising an orally administered controlled release product comprising at least one of EDTA disodium dihydrate and hydroxypropylmethylcellullose for the treatment and/or prevention of disease associated with calcification.

78. A composition comprising of an orally administered controlled release product comprising at least one of EDTA disodium dihydrate and hydroxypropylmethylcellullose for the treatment and/or prevention of disease associated nanobacteria calcifying nanoparticles.
